(19) United States
(12) Patent Application Publication
(10) Pub. No.: US 2019/0129470 A1
(43) Pub. Date: May 2, 2019

(54) MOBILE ELECTRONIC DEVICE

(71) Applicant: SEIKO EPSON CORPORATION, Tokyo (JP)

(72) Inventors: Hironori HASEI, Azumino-shi (JP); Takehisa UEMURA, Shiojiri-shi (JP); Noriaki HIRAIDE, Shiojiri-shi (JP)

(73) Assignee: SEIKO EPSON CORPORATION, Tokyo (JP)

(21) Appl. No.: 16/174,760

(22) Filed: Oct. 30, 2018

(30) Foreign Application Priority Data
Oct. 31, 2017 (JP) 2017-210112

(51) Int. Cl. G06F 1/16; H02S 10/40; H02S 40/38; A61B 5/00; A61B 5/0205

(52) U.S. Cl. CPC G06F 1/1635; H02S 10/40; G06F 1/163; G06F 1/1698; G06F 1/1684; H02S 40/38; A61B 5/02416; A61B 5/7425; A61B 5/7475; A61B 5/0205; A61B 2560/0214; A61B 2562/0219; A61B 5/681

(57) ABSTRACT

A mobile electronic device includes housing that is worn on a user's body; a solar-light power generation unit that has a first and second cell that are arranged with a gap in between; a display unit; a plurality of buttons on which an operation by user is applied; a circuit board is electrically connected to solar-light power generation unit and overlaps, in a plan view, with at least one portion of first area that is configured by connecting the plurality of buttons with straight lines; a wiring unit that is positioned in a second area which is different from first area and includes the gap; a biological sensor positioned in a position that overlaps with first area, and user's biological signal; and wiring unit that electrically connects between solar-light power generation unit positioned in a second area that includes the gap, which is different from first area, and circuit board.

### CROSS-REFERENCES TO RELATED APPLICATIONS

This application claims priority to Japanese Patent Application No. 2017-210112, filed Oct. 31, 2017, the entirety of which is herein incorporated by reference.

### BACKGROUND

### 1. Technical Field

The present invention relates to a mobile electronic device.

### 2. Related Art

In the related art, a mobile electronic device, such as a wearable device (a wrist device), is known that is worn on a human body portion, such as a wrist, using a band or the like and that has a time display function or a position display function. For example, JP-T-2016-530626 discloses a wearable device that is worn on a body of a wearer, as an example of a mobile electronic device, and capable of performing position measuring using a GPS radio wave from a global positioning system (GPS) as an example of a positioning system, acquisition of biological information for measuring a heartbeat or the like, information communication, or the like.

However, in the wearable device that is disclosed in JP-T-2016-530626, because the position measuring, the heartbeat measuring, the information communication, or the like is performed, for example, an amount of consumption of internal power such as power from a secondary battery is increased and the frequency of charging is increased. To charge the battery, a user needs to attach the device to an accessory case or the like. The charging requires much time and effort. There is a detect in that a duration for which the position measuring or the measurement cannot be performed Occurs. Accordingly, devices that are designed to make improvements through the use of a self-power-generation method are also present, but in this case, there is a problem in that the number of constituent elements of the device increases and in that miniaturization and thinning are difficult to achieve.

### SUMMARY

An advantage of some aspects of the invention is to solve at least a part of the problems described above, and the invention can be implemented as the following aspects or application examples.

### APPLICATION EXAMPLE 1

A mobile electronic device according to this application example includes: a housing that is worn on a body of a user; a solar-light power generation unit that is accommodated in the housing and has a first cell and a second cell that are arranged with a gap in between; a display unit that is accommodated in the housing; a plurality of buttons on which an operation by the user is applied; a circuit board that is accommodated in the housing, is electrically connected to the solar-light power generation unit and overlaps, in a plan view in a direction normal to a light reception surface of the first cell, with at least one portion of a first area that is configured by connecting the plurality of buttons with straight lines; a wiring unit that is positioned in a second area which is different from the first area in the plan view and includes the gap; a biological sensor that is accommodated in the housing, is positioned in a position that overlaps with the first area, and detects a biological signal of the user; and a wiring unit that electrically connects between the solar-light power generation unit which is positioned in a second area which is different from the first area in the plan view and includes the gap, and the circuit board.

In the mobile electronic device according to the application example, with power that is generated by the solar-light power generation unit that has at least the first cell and the second cell which are arranged with the gap in between, position measuring, heartbeat measuring, information communication, or the like can be performed. Because of this, under various use environments, power to use can also be provided by self-power-generation, or the frequency of charging can also be decreased. In addition, in the plan view, the circuit board and the biological sensor are arranged in the first area that is an area that is defined by connecting the buttons with straight lines, and the wiring unit is positioned in the second area. Because of this, miniaturization and thinning can be realized.

### APPLICATION EXAMPLE 2

It is preferable that the mobile electronic device according to the application example further includes a light transmitting member that is positioned on the light reception surface side of the solar-light power generation unit, and has a mark which overlaps with the gap in the plan view, in which the gap, the mark and the wiring unit overlap with one another in the plan view.

According to this application example, the gap between the first cell and the second cell, the mark that is provided on the light transmitting member, and the wiring unit are arranged to overlap with one another in the second area. Thus, a width (an area) or a space (a volume) of the first area which the circuit board is positioned can be sufficiently secured. Because of this, the miniaturization and the thinning can be realized.

### APPLICATION EXAMPLE 3

In the mobile electronic device according to the application example, it is preferable that the mark includes at least any one of a letter, a symbol, and a line.

According to this application example, for example, a logo mark, a symbol representing a function of the button, a line, or the like can be easily displayed. Furthermore, with the mark, the gap between the first cell and the second cell can be difficult to visually recognize.

### APPLICATION EXAMPLE 4

In the mobile electronic device according to the application example, it is preferable that the mark has a shape that includes at least a planar shape of the gap in the plan view.

According to this application example, the shape that includes at least the planer shape of the gap between the first cell and the second cell, in other words, with the mark in a shape that overlaps with the gap in the plan view, the gap between the first cell and the second cell in the plan view can be hidden and the gap can be difficult to visually recognize.

### APPLICATION EXAMPLE 5

In the mobile electronic device according to the application example, it is preferable that the solar-light power generation unit overlaps with an outer edge of the display unit in the plan view.

According to this application example, an effective area of the display unit can be secured, and an amount of display can be increased. Along with this, an area of the solar-light power generation unit can be secured and an amount of power generation can be increased.

### APPLICATION EXAMPLE 6

It is preferable that the mobile electronic device according to the application example further includes an antenna unit that is accommodated in the housing, is electrically connected with the circuit board, and receives a wireless signal, and the antenna unit and the solar-light power generation unit overlap with each other in the plan view.

According to this application example, the antenna unit and the solar-light power generation unit are arranged to overlap with each other in the plan view. Thus, a light reception surface of the solar-light power generation unit can be increased and a larger amount of generated power can be obtained.

### APPLICATION EXAMPLE 7

It is preferable that the mobile electronic device according to the application example further includes an antenna unit that is accommodated in the housing, is electrically connected to the circuit board, and receives a wireless signal, and the antenna unit is positioned between an internal wall surface of the housing and the solar-light power generation unit in the plan view.

According to this application example, when viewed from above, the antenna unit is positioned between the inner wall surface of the housing and the solar-light power generation unit, and thus the receiver sensitivity of the antenna unit can be secured.

### APPLICATION EXAMPLE 8

It is preferable that the mobile electronic device according to the application example further includes an antenna unit that is accommodated in the housing, is electrically connected to the circuit board, and receives a wireless signal, and the antenna unit is positioned in the second area.

According to this application example, the antenna unit is positioned in the second area that is different from the first area that is surrounded with the plurality of buttons. Because of this, the receiver sensitivity of the antenna unit can be secured.

### APPLICATION EXAMPLE 9

It is preferable that the mobile electronic device according to the application example further includes an acceleration sensor that detects a body motion of the user, and the acceleration sensor is positioned in the second area.

According to this application example, because a volume of an element of the acceleration sensor is small, the acceleration sensor can also be arranged in the second area in which the solar-light power generation unit or the wiring unit is arranged, and thus, has a comparatively small amount of room for space. Moreover, the acceleration sensor is positioned in the second area that is close to an outer edge portion of the housing, and thus a user operation such as tapping is easy to detect.

### APPLICATION EXAMPLE 10

A mobile electronic device according to this application example includes: a housing that is worn on a body of a user; a solar-light power generation unit that is accommodated in the housing and has a first cell and a second cell that are arranged with a gap in between; a biological sensor that detects a biological signal of the user; a display unit that is accommodated in the housing, and on which biological information on the user, which is derived based on a signal from the biological sensor, is displayed; and a circuit board that is accommodated in the housing, and is electrically connected to the solar-light power generation unit, the biological sensor, and the display unit, in which the gap in the solar-light power generation unit and the biological sensor do not overlap with each other in a plan view in a direction normal to a light reception surface of the first cell.

In the mobile electronic device according to the application example, with power that is generated by the solar-light power generation unit that has at least the first cell and the second cell which are arranged with the gap in between, position measuring, heartbeat measuring, information communication, or the like can be performed. Because of this, under various use environments, power to use can also be provided by self-power-generation, or the frequency of charging can also be decreased. In addition, the biological sensor is positioned in a position that does not overlap with the gap between the first cell and the second cell in the plan view. Because of this, the mobile electronic device can be thinned.

### APPLICATION EXAMPLE 11

In the mobile electronic device according to the application example, it is preferable that, in a cross sectional view in a direction orthogonal to a direction normal to the light reception surface of the first cell and, the display unit is positioned between the solar-light power generation unit and the circuit board.

According to this application example, the user can visually recognize displaying on the display unit in an easy manner without the displaying being blocked by the circuit board. Furthermore, because an outer edge of the display unit can be hidden in the solar-light power generation unit, an aesthetical property can be improved.

### APPLICATION EXAMPLE 12

In the mobile electronic device according to the application example, it is preferable that, in the cross sectional view, the circuit board is positioned between the biological sensor and the solar-light power generation unit.

According to this application example, so-call stray light that refers to incident light for power generation toward the solar-light power generation unit, which becomes leakage light entering from the gap between the first cell and the second cell, any other gap, or the like, and enters a package from the solar battery side, can be blocked by the circuit board, and thus an influence of external light on measurement by the biological sensor can be reduced.

### APPLICATION EXAMPLE 13

It is preferable that the mobile electronic device according to the application example further includes a secondary battery that supplies power to at least the circuit board and the biological sensor, and in the cross sectional view, the secondary battery is positioned between the biological sensor and the circuit board.

According to this application example, because a distance between the solar-light power generation unit and the biological sensor can be secured with the positioning of this secondary battery, an influence on the measurement by the biological sensors due to the stray light that refers to the incident light for power generation toward the solar-light power generation unit, which becomes the leakage light entering from the gap between the first cell and the second cell, any other gap, or the like and enters a package from the solar-light power generation unit, can be reduced.

### APPLICATION EXAMPLE 14

It is preferable that the mobile electronic device according to the application example further includes a plurality of buttons that are connected to the circuit board and on which an operation of the user is applied; and a wiring unit that electrically connects between the circuit board and the display unit and is positioned to include the gap in the plan view, and in the plan view, the circuit board has a first area that is configured by connecting the plurality of buttons with straight lines, and the biological sensor is positioned in the first area, and, in the plan view, the wiring unit is positioned in a second area that is different from the first area.

According to this application example, the circuit board and the biological sensor are arranged in the first area that is an area that is defined by connecting the buttons with straight lines, and the wiring unit is positioned in the second area. Because of this, the miniaturization and the thinning can be realized.

### APPLICATION EXAMPLE 15

In the mobile electronic device according to the application example, it is preferable that an acceleration sensor that detects a body motion of the user is positioned in the second area.

According to this application example, because a volume of an element of the acceleration sensor is small, the acceleration sensor can also be arranged in the second area in which the solar-light power generation unit or the wiring unit is arranged and thus has a comparatively small amount of room for space. Moreover, the acceleration sensor is positioned in the second area that is close to an outer edge portion of the housing, and thus, a user operation such as tapping is easy to detect.

### BRIEF DESCRIPTION OF THE DRAWINGS

The invention will be described with reference to the accompanying drawings, wherein like numbers reference like elements.

FIG. 1 is a schematic configurational diagram illustrating an outline of an exercise assistance system in which a wrist device as a mobile electronic device finds application.

FIG. 2 is a perspective diagram for external appearance illustrating a schematic configuration of the wrist device according to a first embodiment, when viewed from the front surface side (the display surface side).

FIG. 3 is a perspective diagram for external appearance illustrating the schematic configuration of the wrist device according to the first embodiment, when viewed from the rear surface side.

FIG. 4 is a cross-sectional diagram illustrating a configuration of the wrist device according to the first embodiment.

FIG. 5 is a plan view diagram illustrating the configuration of the wrist device according to the first embodiment.

FIG. 6 is a functional block diagram illustrating the schematic configuration of the wrist device according to the first embodiment.

FIG. 7 is a cross-sectional diagram illustrating a configuration of a wrist device according to a second embodiment.

FIG. 8 is a plan view diagram illustrating the configuration of the wrist device according to the second embodiment.

### DESCRIPTION OF EXEMPLARY EMBODIMENTS

Systems according to embodiments of the invention will be described below. It is noted that the embodiments which will be described below do not improperly limit the subject matters of the invention, which are recited by the appended claims. Furthermore, all configurations that will be described in each embodiment are not limited to being configurational requirements for the invention.

### 1. Techniques According to the Present Embodiment

First, as an example of a system in which a mobile electronic device according to the invention finds application, an exercise assistance system will be described. As an example of the mobile electronic device, a wrist device (a wearable device) that, for example, includes a pulse wave sensor or a body motion sensor which is worn on a wrist of a user will be described below in an exemplary manner.

The wrist device as the mobile electronic device that is used for the exercise assistance system includes a solar-light power generation unit on the display unit side, and includes the pulse wave sensor that acquires pulse wave information as biological information on the user or the body motion sensor that acquires motion information on the user. Moreover, the wrist device includes a global positioning system (GPS) as an example of a positioning system that uses a positional information satellite which is called a global navigation satellite system (GNSS) or the like that acquires positional information on the user. It is noted that the mobile electronic device is not limited to the wrist device and may be a wearable device that is worn on any other body portion of the user, such as the neck or the ankle.

It is possible that a pulse wave sensor as an example of a biological sensor that detects a biological signal of the user acquires the pulse wave information such as the number of pulses. As the pulse wave sensor, for example, a photoelectric sensor (an optical sensor) is used. In this case, a technique is considered that, with the photoelectric sensor, detects reflection light or transmission light which results from emitting light to a living body. An amount of emitted light absorbed in the living body and an amount of emitted light reflected from the living body vary according to an amount of blood flowing through a blood vessel. Because of this, sensor information that is detected in the photoelectric sensor is a signal that corresponds to the amount of flowing blood or the like. Therefore, information relating to a heartbeat can be acquired by interpreting the signal. However, the pulse wave sensor is not limited to the photoelectric sensor, and any other sensor, such as an electrocardiograph or an ultrasonic sensor, may be used as the pulse wave sensor.

It is noted that the photoelectric sensor (the optical sensor) needs to receive necessary light and block unnecessary light, and that in the case of an example of the pulse wave sensor, the photoelectric sensor needs to receive the reflection light which includes a pulse wave component that is reflected from a subject (particularly, a human body portion in which a blood vessel that is a measurement target is included) which is a target object for measurement and to block other light because the light, if not blocked, becomes a noise component.

The body motion sensor is a sensor that detects a body motion of the user. As the body motion sensor, an acceleration sensor, an angular speed sensor, a direction sensor (a geomagnetic sensor), a pressure sensor (an altitude sensor), or the like is considered, but other sensors may be used.

The GPS is also a global navigation satellite system, and is a satellite positioning system for measuring a current position on the earth based on a plurality of satellite signals. The GPS has a function of acquiring the positional information on the user for performing positioning calculation using GPS time information and orbital information and a function of correcting the time in a watch function.

### 2. Exercise Assistance System

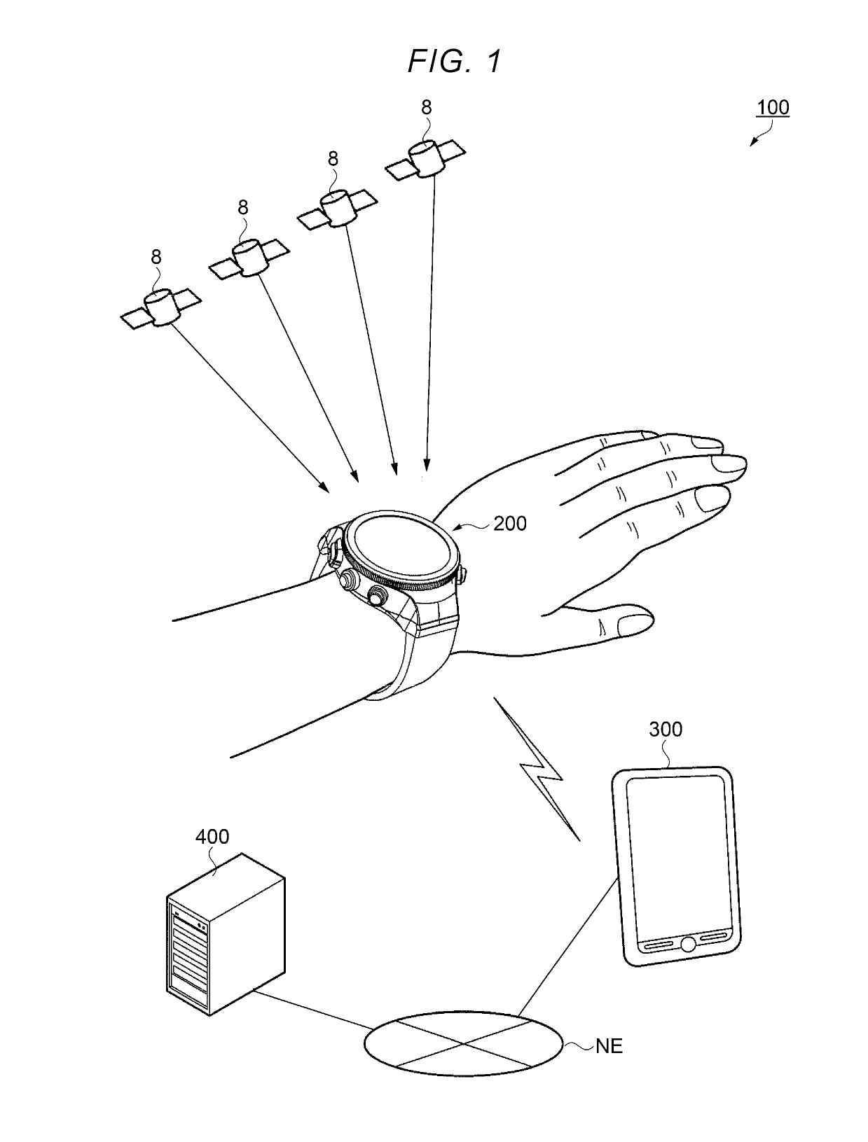

Next, a configuration of the exercise assistance system in which the wrist device as the mobile electronic device finds application will be described below with reference to FIG. 1. FIG. 1 is a schematic configurational diagram illustrating an outline of the exercise assistance system in which the wrist device as the mobile electronic device finds application.

An exercise assistance system 100 according to the present embodiment, as illustrated in FIG. 1, includes a wrist device 200 as the mobile electronic device that is a detection device which includes the pulse wave sensor as the biological sensor (the photoelectric sensor), the acceleration sensor as the body motion sensor, the GPS, and the like, a mobile device 300 as an exercise assistance device, and a server 400 as an information processing apparatus that is connected to the mobile device 300 via a network NE.

The GPS as the global navigation satellite system that is included in the wrist device 200 has a function of receiving a radio wave (a satellite signal) from GPS satellite and correcting the internal time, or of acquiring the positional information for performing the positioning calculation. The GPS satellite 8 is an example of the positional information satellite that travels in a prescribed orbit around the earth, and transmits a high frequency radio wave on which a navigation message is superimposed, toward the earth. In the following description, the radio wave on which the navigation message is superimposed is referred to as the satellite signal.

The satellite signal from the GPS satellite 8 includes extreme-precision GPS time information and a time compensation parameter for compensating for a time error. The wrist device 200 receives the satellite signal (the radio wave) that is transmitted from one GPS satellite 8, and can acquire time information using the GPS time information and the time compensation parameter, which are included in the satellite signal.

Furthermore, the orbital information indicating a position of the GPS satellite 8 in orbit is also included in the satellite signal. The wrist device 200 can perform the positioning calculation using the GPS time information and the orbital information. The positioning calculation is performed on the assumption that the internal time of the wrist device 200 has an error to some degree. That is, a time error, as well as x, y, and z parameters for specifying a three-dimensional position of the wrist device 200, is unknown. For this reason, the wrist device 200, for example, receives the satellite signal (the radio wave) that is transmitted from each of three or more GPS satellites 8, and performs the positioning calculation using the GPS time information and the orbital information that are included in the satellite signal, and thus can acquire the positional information on a current place.

The mobile device 300 as the exercise assistance device, for example, can be configured with a smartphone, a tablet-type terminal device, or the like. The mobile device 300 is connected to the wrist device 200 in which the photoelectric sensor (the pulse wave sensor) as the biological sensor or the acceleration sensor as the body motion sensor is used, for example, through a short distance wireless communication, a wired communication (not illustrated), or the like, of which an example can be Bluetooth (a registered trademark) communication or the like. The mobile device 300 receives measurement information from the wrist device 200, and can report the pulse wave information or body motion information on the user, which is processed, the positional information, or the like. However, various modifications to the mobile device 300 are possible. For example, a photoelectric sensor unit 40, a body motion sensor unit 170, a GPS reception unit 160, or the like that is included in the wrist device 200, which will be described below, may be included in the mobile device 300.

It is noted that the wrist device 200 and the mobile device 300 according to the present embodiment have a Bluetooth function, and the mobile device 300 and the wrist device 200 are connected to each other, for example, using Bluetooth low energy (which is also referred to as Bluetooth 4.0) for Bluetooth communication. With the Bluetooth Low Energy, in which the power saving feature is regarded as important, a significant amount of power saving is achieved when compared with the earlier version, and it is possible that the time available for use of the wrist device is increased.

Furthermore, the mobile device 300 can be connected to a personal computer (PC) or the server 400 such as a server system through the network NE. Regarding the network NE here, various networks NE can be used such as a wide area network (WAN), a local area network (LAN), a mobile telephone communication network, and short distance wireless communication. In this case, the server 400 is realized as a processing storage unit that receives and stores the pulse wave information or the body motion information that is measured in the wrist device 200, or data that is processed in the mobile device 300, from the mobile device 300 through the network NE.

It is noted that, according to the embodiment as described above, it suffices that the wrist device 200 is capable of communicating with the mobile device 300 and wrist device 200 does not need to be connected directly to the network NE. Consequently, it is possible that the configuration of the wrist device 200 is simplified. However, modifications to the exercise assistance system 100 are also possible in which the mobile device 300 is omitted and in which a direct connection is made between the wrist device 200 and the server 400. In this case, the wrist device 200 has a function of processing the measurement information that is included in the mobile device 300, and a function of transmitting the measurement information to the server 400 or receiving the measurement information from the server 400.

Furthermore, the exercise assistance system 100 is not limited to being realized with a configuration in which the server 400 is included. For example, processing or a function that is performed in the exercise assistance system 100 may be realized with the mobile device 300. For example, in most cases, the mobile device 300 such as a smartphone has a limitation on processing performance, a storage area, or a battery capacity when compared with the server system, but taking into account a recent improvement in performance, it is also considered that sufficient processing performance or the like is possible. Consequently, if a requirement for processing performance or the like is satisfied, it is possible that the processing or the function which is performed in the exercise assistance system 100 according to the present embodiment is realized in the mobile device 300 alone.

Furthermore, the exercise assistance system 100 according to the embodiment is not limited to being realized with the wrist device, the mobile device, and the server. For example, the exercise assistance system 100 may include two or more of the wrist device 200, the mobile device 300, and the server 400. In this case, the processing that is performed in the exercise assistance system 100 may be performed in any one of the wrist device 200, the mobile device 300, and the server 400, or may be distribution-processed among the wrist device 200, the mobile device 300, and the server 400. Furthermore, although the exercise assistance system 100 according to the present embodiment includes a device and an apparatus other than the wrist device 200, the mobile device 300, and the server 400, this does not pose any problem. Moreover, in a case where an improvement in terminal performance or a type of usage, or the like is considered, an embodiment that realizes the exercise assistance system 100 according to the present embodiment with the wrist device 200 can be set to be employed.

Furthermore, the exercise assistance system 100 according to the present embodiment includes a memory in which information (for example, a program or various pieces of data) is stored, and a processor that operates based on the information which is stored in the memory. A function of each of the units of the processor, for example, may be realized in individual software or may be realized in integrated hardware. The processor, for example, may be a central processing unit (CPU). However, the processor is not limited to the CPU, and it is possible that various processors are used such as a graphics processing unit (GPU) and a digital signal processor (DSP). Furthermore, the processor may be a hardware circuit that is an ASIC. The memory, for example, may be a semiconductor memory, such as a static random access memory (SRAM) or a dynamic random access memory (DRAM), may be a register, may be a magnetic storage device, such as a hard disk device, and may be an optical storage device, such as an optical disk. For example, a computer-readable instruction is stored in the memory, and the instruction is executed by the processor, and thus the function of each of the units of the exercise assistance system 100 is realized. The instruction here may be an instruction that constitutes a program, and may be an instruction for instructing a hardware circuit of the processor to operate.

### 3. Wrist Device According to a First Embodiment

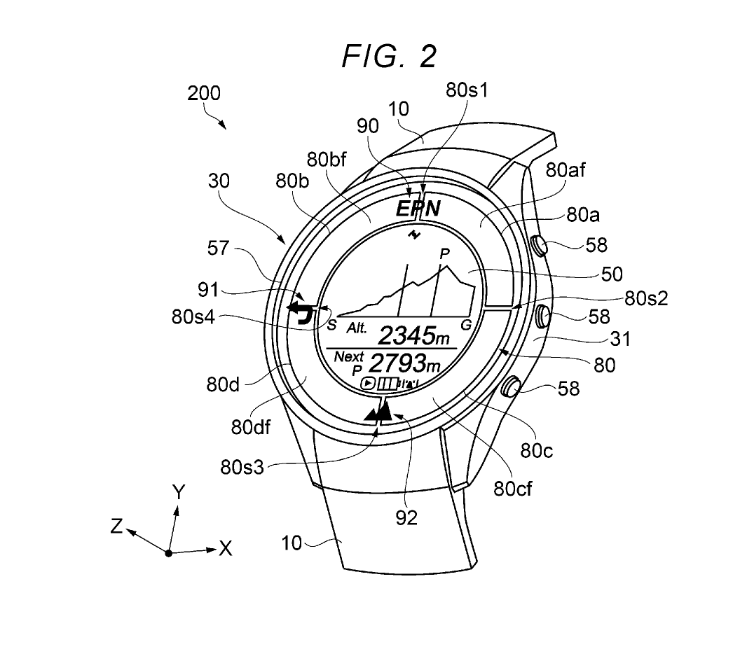

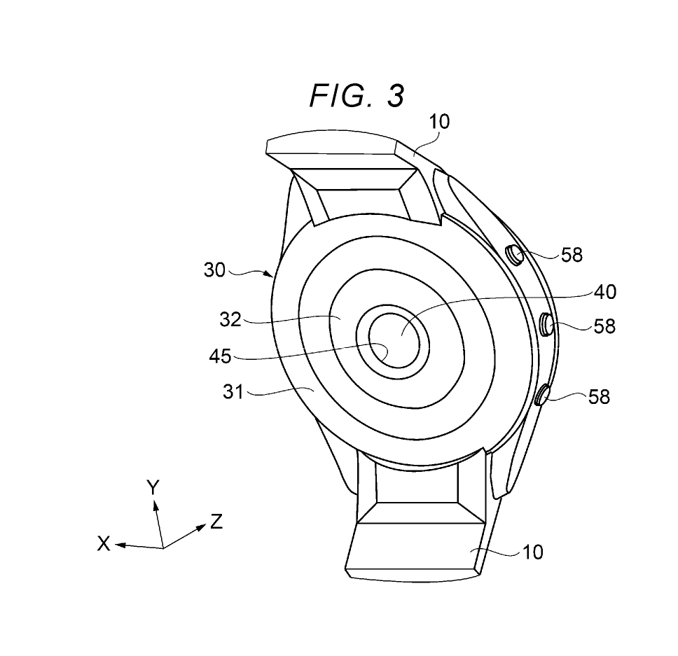

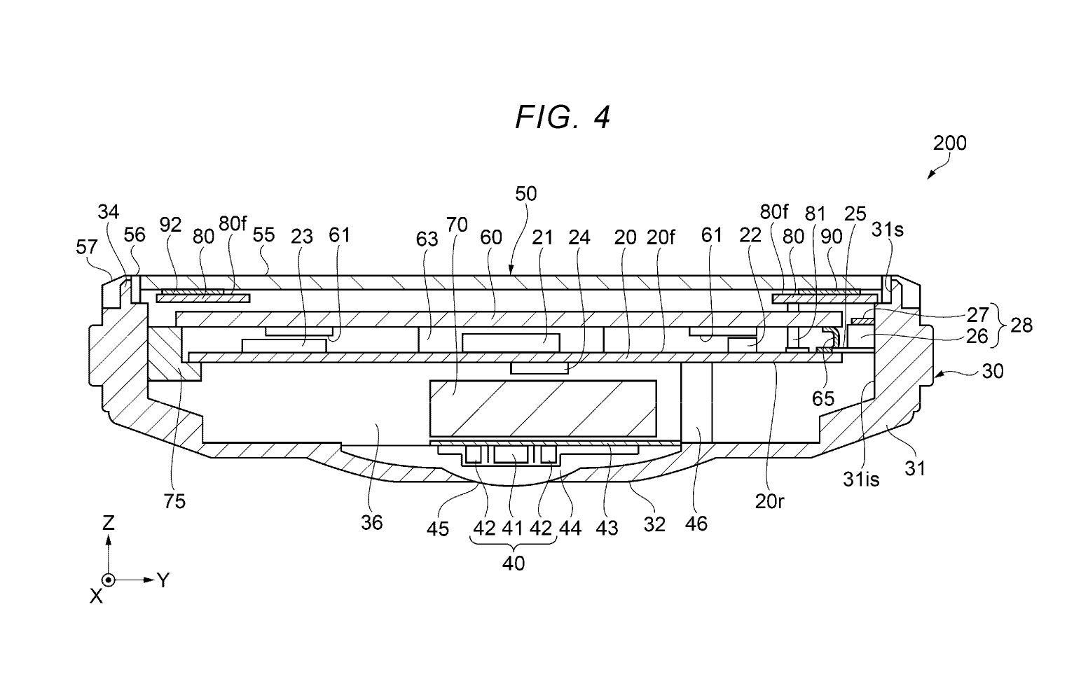

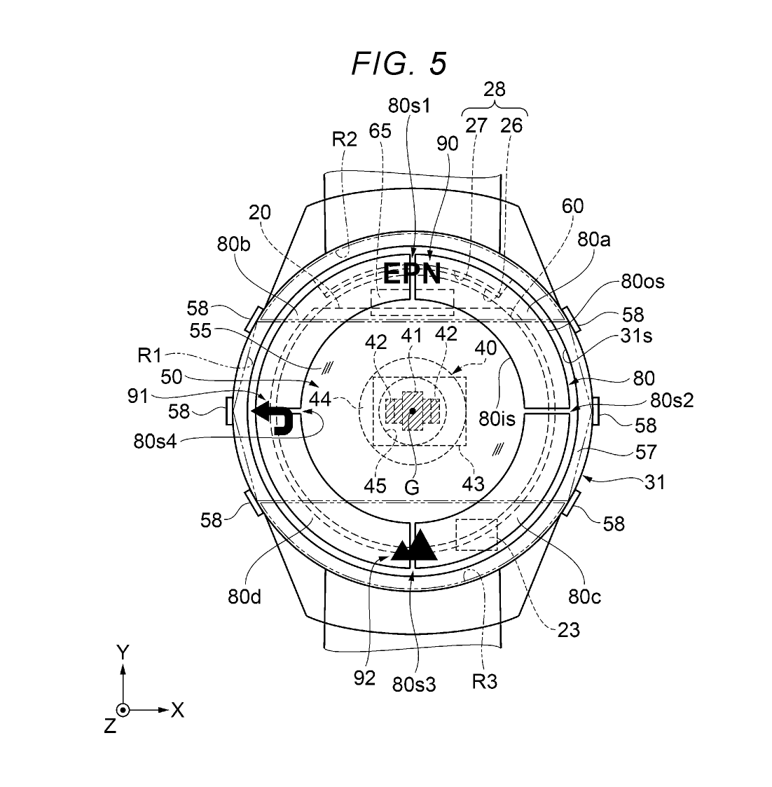

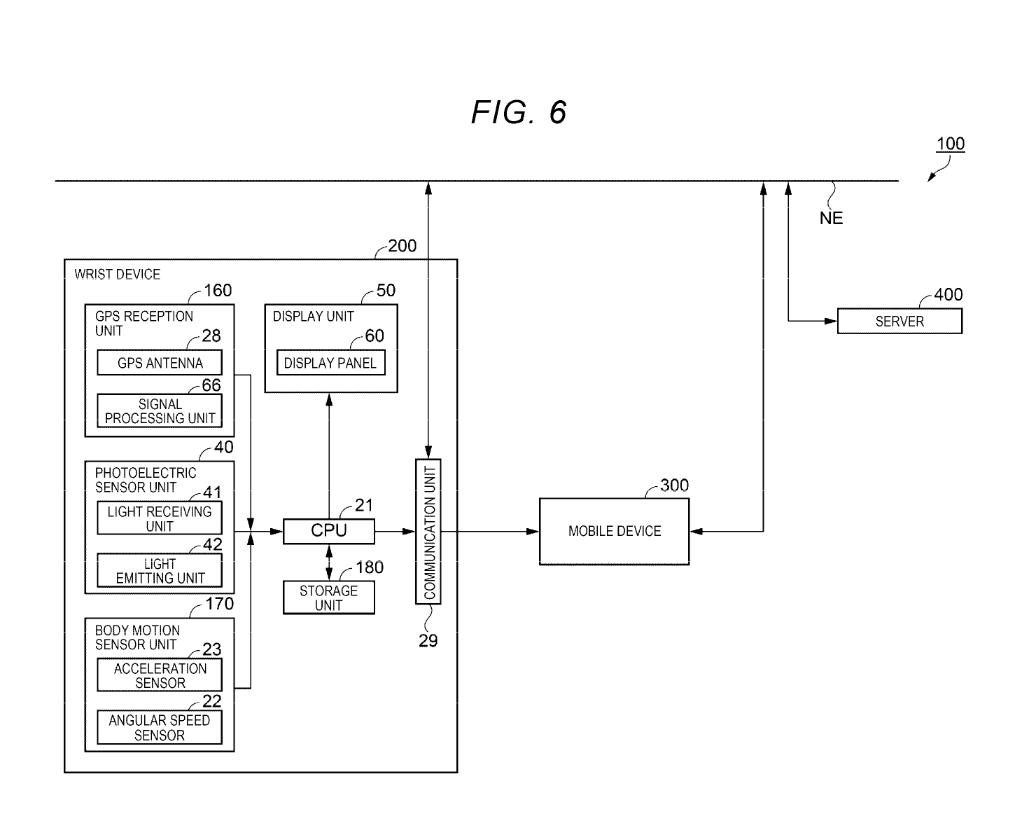

Next, a configuration of a wrist device (a measurement device) as the mobile electronic device according to a first embodiment will be described with reference to FIGS. 2, 3, 4, 5 and 6. FIG. 2 is a perspective diagram for external appearance illustrating a schematic configuration of the wrist device according to the first embodiment, when viewed from the front surface side (the display surface side). FIG. 3 is a perspective diagram for external appearance illustrating the schematic configuration of the wrist device according to the first embodiment, when viewed from the rear surface side. FIG. 4 is a cross-sectional diagram illustrating a configuration of the wrist device according to the first embodiment. FIG. 5 is a plan view diagram illustrating the configuration of the wrist device according to the first embodiment. FIG. 6 is a functional block diagram illustrating the schematic configuration of the wrist device according to the first embodiment.

It is noted that in a description of a wrist device 200 according to the first embodiment, a side of the device main body 30, which is positioned toward a target object that is a target human body portion for measurement to obtain biological information or the like when a device main body 30 is worn on the user, is described as a “rear side or a rear surface side,” and the display surface side of the device main body 30, which is the opposite side, is described as a “front side or a front surface side.” Furthermore, in some cases, a “target object (a target human body portion)” is referred to as a “subject.” Furthermore, a coordinate system is set with a housing 31 of the wrist device 200 serving as a reference, the center of a display surface of a display unit 50 is set to be the origin, and one part of a direction that intersects the display surface of the display unit 50, that is, a direction from the rear surface to the front surface in a case where the display surface side of the display unit 50 is defined as the front surface is set to be the Z-axis positive direction (the +Z-axis direction). Alternatively, a direction normal to the display surface of the display unit may be the Z-axis positive direction. Alternatively, a direction from the photoelectric sensor unit 40 that constitutes the pulse wave sensor which is an example of the biological sensor that detects the biological signal on the user, to the display unit 50, or one part of a direction normal to light reception surfaces 80af, 80bf, 80cf, and 80df of panel type cells (a first cell 80a, a second cell 80b, a third cell 80c, and a fourth cell 80d) that constitute a solar-light power generation unit 80, that is, a direction away from the housing 31 may be defined as the Z-axis positive direction. The Z-axis positive direction in a state where the wrist device 200 is worn on the subject is equivalent to a direction from the subject to the housing 31. Furthermore, two axes orthogonal to the Z-axis are set to be the X and Y axes, and a direction in which a band portion 10 is attached particularly to the housing 31 is set to be the Y-axis. It is noted that the light reception surfaces 80af, 80bf, 80cf, and 80df are surfaces of the first cell 80a, the second cell 80b, the third cell 80c, and the fourth cell 80d that constitute the solar-light power generation unit 80 on which light is incident.

FIG. 2 is a perspective diagram illustrating the wrist device 200 the band portion 10 of which is in a fixed state, when viewed from the +Z-axis direction that is a direction toward the front side (the display unit 50 side), which is opposite in direction to the rear side that is the subject side in a worn state. Furthermore, FIG. 3 is a perspective diagram when viewed from the −Z-axis direction toward the rear side, which is opposite in direction to the +Z-axis direction in FIG. 2. Furthermore, FIG. 4 is a cross-sectional diagram when viewed from the +X-axis direction. Furthermore, FIG. 5 is a plan-view diagram when viewed from the +Z-axis direction.

The wrist device 200 as the mobile electronic device, as illustrated in FIGS. 2, 3, and 4, is worn on a given human body portion (for example, a target human body portion for measurement, such as the wrist) of the user, and detects the pulse wave information or the body motion information, the positional information, and the like. The wrist device 200 has the device main body 30 that includes the housing 31, which is brought into close contact with the user to detect the pulse wave information, the body motion information, or the like, and a pair of band portions 10 that are attached to the device main body 30 to bring the device main body 30 into close contact with the user.

The device main body 30 including the housing 31 includes the display unit 50, the solar-light power generation unit 80 in the shape of a ring, which includes the light reception surfaces 80af, 80bf, 80cf, and 80df of the cells (the first cell 80a, the second cell 80b, the third cell 80c, and the fourth cell 80d), respectively, which are positioned on an outer edge portion of the display unit 50 toward the +Z-axis direction, and a measurement window portion 45 that corresponds to the photoelectric sensor unit 40 (refer to FIG. 4) as the biological sensor. It is noted that, in a plan view in the +Z-axis direction (the direction normal to the light reception surfaces 80af, 80bf, 80cf, and 80df), the display unit 50 and the solar-light power generation unit 80 are arranged in such a manner as to partly overlap with each other. Furthermore, a plurality of operation units (operation buttons) 58 that are connected to a circuit board 20 (refer to FIG. 4) are provided on an external flank surface of the device main body 30, and a bezel 57 is provided that is positioned in a such a manner as to surround the outer edge portion of the display unit 50 in a ring-shaped manner. However, the wrist device 200 is not limited to this configuration, and various modifications are possible such as omission of one or several of these constituent elements and addition of other constituent elements. It is noted that the outer edge portion of the display unit 50 means an outer circumference of a liquid crystal display (a display panel 60) that constitutes the display unit 50, or an area that surrounds a display area on which information can be displayed.

It is noted that in the following description, as illustrated in FIG. 5, in the plan view in the +Z-axis direction, an area that is surrounded with the plurality of operation units (operation buttons) 58 that are connected to the circuit board 20 (refer to FIG. 4), for example, an area that is the center portion of the housing 31, which is defined by connecting protrusion portions that protrude toward sides of the plurality of operation units (operation buttons) 58, which face an external flank surface of the device main body 30, with straight lines, is defined as a first area R1. Furthermore, areas that are different from the first area R1, which overlap with the housing 31, are defined as second areas R2 and R3. In this configuration, an area that is positioned in the +Y-axis direction (toward the twelve o'clock side) with respect to the first area R1 is expressed as a “second area R2,” and an area that is positioned in the −Y-axis direction (toward the six o'clock side) with respect to the first area R1 is expressed as a “second area R3.” It is noted that in the present specification, each of the first area and the second areas R2 and R3 means a planar area and a spatial area within the housing 31. Furthermore, each of the first area and the second areas R2 and R3 can also be defined as an area that is surrounded with a plurality of tact switches (not illustrated) which are arranged on the circuit board 20, although this is not given as an example in the description of the present embodiment.

The device main body 30 includes the housing 31 that includes an opening portion 31s which is open to the front side. Regarding the rear side of the housing 31, the measurement window portion 45 of the photoelectric sensor unit 40 is provided on a top of protrusion portion 32 that protrudes from a rear surface, the rear side surface, of the housing 31. Then, the photoelectric sensor unit 40 as the biological sensor is positioned at a position that corresponds to the measurement window portion 45 in the plan view in the +Z-axis direction, and a transparent cover 44 is inserted into the measurement window portion 45. It is noted that the transparent cover 44 may protrude from the top of the protrusion portion 32. It is noted that it can be said that, in the plan view in the +Z-axis direction, the measurement window portion 45 of the photoelectric sensor unit 40 is positioned within the first area R1. In this manner, the photoelectric sensor unit 40 (the measurement window portion 45) is positioned in the first area R1, more precisely, in the center portion of the housing 31, which is in a position that does not overlap with a gap between the first cell 80a and the second cell 80b in the plan view, and thus the housing 31 (the wrist device 200 as the mobile electronic device) can be thinned. Furthermore, the photoelectric sensor unit 40 (the measurement window portion 45) is positioned in the center portion of the housing 31, and thus, a balance of the device main body 30 can be improved, and a property of being wearable on the user can be improved.

It is noted that the housing 31 can be formed, for example, of metal such as stainless steel, resin, or the like. It is noted that a configuration of the housing 31 is not limited to a one-piece configuration, and may be a configuration in which the housing 31 is divided into a plurality of parts, for example, a two-piece structure configuration in which a rear cover of the housing 31 is provided toward a direction in which the wrist device is worn on the user.

Regarding the device main body 30, the bezel 57 is provided on the outer circumference side of a protrusion portion 34 that is provided, in an upright protruding manner in the +Z-axis direction, on an outer edge (refer to FIG. 4) of the opening portion 31s of the housing 31 that is positioned on the front side of the device main body 30. Along with this, a windshield (a glass plate in the present example) 55 that is a light transmitting member as a top-plate portion which protects an internal structure is provided inside of the bezel 57. The windshield 55 as the light transmitting member is positioned in such a manner as to close an opening in the housing 31 in a direction toward the light reception surfaces 80af, 80bf, 80cf, and 80df of the solar-light power generation unit 80, in other words, in the plan view in the +Z-axis direction. The windshield 55 is attached to the inner edge side of the protrusion portion 34 of the housing 31 with a joining member 56 such as a packing material or adhesive material. Then, an internal space 36 that is a closed space is provided inside of the housing 31 that is surrounded with the housing 31 and the windshield 55 that closes the opening in the housing 31.

It is noted that the windshield 55 is not limited to the glass plate and is the light transmitting member through which the display unit 50 can be read, and that any member, such as transparent plastic, that has a strength capable of protecting a constituent element, such as the liquid crystal display (the display panel 60) that constitutes the display unit 50, which is accommodated in the internal space 36, can be used as a material other than glass. Furthermore, various marks (for example, a first mark 90, a second mark 91, and a third mark 92) each of which includes at least any one of the letter, the symbol, and the line is provided on the rear surface side or the front surface side (the rear surface side in the present embodiment) of the windshield 55.

Then, as illustrated in FIG. 4, for example, the circuit board 20, an angular speed sensor 22 or an acceleration sensor 23 as a sensor that is included in the body motion sensor unit 170 (refer to FIG. 6), a GPS antenna 28 as an antenna unit, the photoelectric sensor unit 40, a liquid crystal display (hereinafter referred to as the display panel 60) that constitutes the display unit 50, a lighting unit 61 of the display panel 60, a secondary battery 70 (a lithium secondary battery), the solar-light power generation unit 80, and the like, which are constituent elements that constitute the wrist device 200, are accommodated in the internal space 36 within the housing 31. However, the device main body 30 is not limited to a configuration that is illustrated in FIG. 4, and for example, other sensors, such as an atmospheric pressure sensor for calculating a height above sea level or the like, and an air temperature sensor for measuring temperature, a vibrator, and the like may be added.

Here, the circuit board 20 is positioned in such a manner as to overlap with at least one portion of the first area in the plan view in the +Z-axis direction. Furthermore, to the circuit board 20 a flexible wiring board 65 as a wiring unit for connections with the constituent elements, for example, a wiring unit that electrically connects between the circuit board 20 and the display panel 60 that constitutes the display unit 50, a central processing unit (CPU) 21 as a processing unit that includes a control circuit which controls each of the sensors, the display unit 50, and the like that constitute the wrist device 200, a drive circuit, and the like, and other circuit elements 24 are connected. It is noted that the CPU 21 as the processing unit is electrically connected to each of the sensors, for example, the photoelectric sensor unit 40 or the acceleration sensor 23, and can process a signal that is detected by each of the sensors. Furthermore, the CPU 21 is electrically connected to the display panel 60 that constitutes the display unit 50, and can control display on the display panel 60. Furthermore, the angular speed sensor 22 or the acceleration sensor 23 may be connected to the circuit board 20. Furthermore, a plurality of tact switches (not illustrated) with which a plurality of operation units (operation buttons) 58 come into contact can be arranged on the circuit board 20.

Among the constituent elements that constitute the wrist device 200, which are arranged in the internal space 36, the circuit board 20, the photoelectric sensor unit 40, the display panel 60, the secondary battery 70, and the solar-light power generation unit 80 are arranged toward the −Z-axis direction from the windshield 55 side, in the following order: the solar-light power generation unit 80, the display panel 60, the circuit board 20, the secondary battery 70, and the photoelectric sensor unit 40. In this manner, within the housing 31, the display panel 60 that constitutes the display unit 50 is positioned between the solar-light power generation unit 80 and the circuit board 20, and thus the user can visually recognize displaying on the display unit 50 in an easy manner without the displaying being blocked by the circuit board 20.

The solar-light power generation unit (solar cells) 80 is divided into four cells (the first cell 80a, the second cell 80b, the third cell 80c, and the fourth cell 80d) between the windshield 55 and the display panel 60, and is positioned in such a manner that gaps 80s1, 80s2, 80s3, and 80s4 are present between pairs of the cells, respectively, that are positioned adjacent to each other. Specifically, the gap 80s1 is provided between the first cell 80a and the second cell 80b, the gap 80s2 is provided between the first cell 80a and the third cell 80c, the gap 80s3 is provided between the third cell 80c and the fourth cell 80d, and the gap 80s4 is provided between the second cell 80b and the fourth cell 80d. It is noted that the gap 80s1 between the first cell 80a and the second cell 80b is positioned in such a manner as to be within the second area R2 in the twelve o'clock direction that is the +Y-axis direction. Then, the solar-light power generation unit 80 is positioned in such a manner that the light reception surfaces 80af, 80bf, 80cf, and 80df of four cells (the first cell 80a, the second cell 80b, the third cell 80c, and the fourth cell 80d) face in the +Z-axis direction.

The first mark 90 that is provided on the rear surface side of the windshield 55 is configured with letters “EPN.” The first mark 90 is positioned in the second area R2 and is provided in such a manner as to overlap with the gap 80s1 between the first cell 80a and the second cell 80b. In the same manner, the second mark 91 that is provided on the rear surface side of the windshield 55 is configured with a symbol for an “arrow” representing return. The second mark 91 is positioned in the first area R1 and is provided in such a manner as to overlap with the gap 80s4 between the fourth cell 80d and the second cell 80b. In the same manner, the third mark 92 that is provided on the rear surface side of the windshield 55 is configured with a “pictograph” representing a mountain. The third mark 92 is positioned in the second area R3 and is provided in such a manner as to overlap with the gap 80s3 between the third cell 80c and the fourth cell 80d.

In this manner, for example, a logo mark (for example, the first mark 90), a symbol (for example, the second mark 91) representing a function of the operation unit 58 (the operation button), a line or a picture (a pictograph), or the like (for example, the third mark 92) can be easily displayed. With the first mark 90, the gap 80s1 between the first cell 80a and the second cell 80b can be made difficult to visually recognize. With the second mark 91, the gap 80s4 between the fourth cell 80d and the second cell 80b can be made difficult to visually recognize. Furthermore, with the third mark 92, the gap 80s3 between the third cell 80c and the fourth cell 80d can be made difficult to visually recognize. It is noted that another mark may be provided in such a manner as to overlap with the gaps 80s2, and that in this case, the same effect as described above can be achieved.

Furthermore, it is preferable that, in the plan view in the +Z-axis direction, an outer circumference end portion of the display panel 60 that is electrically connected to the CPU 21 is positioned between an outer circumference 80os that is an outer edge portion in the shape of a circle, of the solar-light power generation unit 80, and an inner circumference 80is in the shape of a circle, which is an inner edge portion. The outer circumference 80os and the inner circumference 80is will be described below. In this manner, in the plan view in the +Z-axis direction, the outer edge end portion of the display panel 60 is positioned between the outer circumference 80os and the inner circumference 80is of the solar-light power generation unit 80. In other words, the outer circumference end portion of the display panel 60 overlaps with the solar-light power generation unit 80, and thus an arrangement balance between the solar-light power generation unit 80 and the display unit 50 (the display panel 60) can be improved, and a wider area of the solar-light power generation unit 80 can be secured while an area of the display unit 50 is secured. Moreover, an end portion of the display unit 50 is positioned in such a manner as to be covered with the solar-light power generation unit 80, and thus the end portion of the display unit 50 can be hidden. Because of this, an aesthetical property can be improved.

Furthermore, within the housing 31, when viewed from a cross section, that is, from a direction (the X-axis direction) orthogonal to a direction (the +Z-axis direction) normal to the light reception surfaces 80af, 80bf, 80cf, and 80df of the solar-light power generation unit 80, the circuit board 20 is positioned between the solar-light power generation unit 80 and the photoelectric sensor unit 40, and thus, so-called stray light, which refers to light incident for power generation toward the solar-light power generation unit 80, which becomes leakage light that enters, for example, from the gap 80s1 between the first cell 80a and the second cell 80b, any other gap, or the like, and enters the housing 31, can be blocked by the circuit board. Accordingly, an influence of external light on the photoelectric sensor unit 40 can be reduced.

Furthermore, when viewed from the cross section, the secondary battery 70 is positioned between the display unit 50 and the photoelectric sensor unit 40 within the housing 31, and thus a distance between the solar-light power generation unit 80 and the photoelectric sensor unit 40 can be secured. Accordingly, an influence on measurement by the photoelectric sensor unit 40, due to so-called stray light, which refers to light incident for power generation toward the photoelectric sensor unit 40, which becomes leakage light that enters, for example, from the gap 80s1 between the first cell 80a and the second cell 80b, any other gap, or the like, and enters the housing 31, can be reduced.

Furthermore, when viewed from the cross section, the display panel 60 that constitutes the display unit 50 is positioned between the solar-light power generation unit 80 and the photoelectric sensor unit 40 within the housing 31, and thus, so-called stray light, which refers to light incident for power generation toward the solar-light power generation unit 80, which becomes leakage light that enters from the gap 80 or the like, and enters the housing 31 from the solar-light power generation unit 80 side, can be blocked by the display panel 60, and thus, an influence of external light (stray light) on the photoelectric sensor unit 40 can be reduced.

The flexible wiring board 65 as the wiring unit electrically connects between the circuit board 20 and the display panel 60. In the plan view in the +Z-axis direction, the flexible wiring board 65 is provided on the second area R2 in the +Y-axis direction. It is noted that in the plan view in the +Z-axis direction, the flexible wiring board 65 is positioned in such a manner as to overlap with the gap 80s1 between the first cell 80a and the second cell 80b.

It is noted that in the plan view in the +Z-axis direction, the flexible wiring board 65 may be positioned in the second area R2 that does not overlap with the gap 80s1 between the first cell 80a and the second cell 80b. In this manner, the flexible wiring board 65 is positioned in the second area R2, and thus a width (an area) or a space (a volume) of the first area R1 in which the circuit board 20 is positioned can be sufficiently secured. Because of this, the miniaturization and the thinning of the housing 31 can be realized.

The GPS antenna 28 as the antenna unit that receives a positioning satellite signal includes a base 26, as a non-conductive member, which is configured, for example, with a resin member, and a conductor 27 that is configured, for example, with metal such as copper, copper alloy, aluminum, or aluminum alloy, which is provided on the windshield 55 side (the +Z-axis direction) surface of the base 26. The base 26, as illustrated in FIG. 5, is positioned in a curved manner along an inner circumference of the opening portion 31s of the housing 31 in the twelve o'clock direction (the +Y-axis direction), and, in the plan view in the +Z-axis direction, is positioned in such a manner as to overlap with the solar-light power generation unit 80 (any one of the cells 80a, 80b, 80c, and 80d). The non-conducting base 26 is determined as a dielectric, and thus wavelength shortening effect due to the base 26 (the dielectric) is used. As a result, the GPS antenna 28 is configured as an antenna. Furthermore, with this arrangement, receiver sensitivity of the GPS antenna 28 can be increased due to capacity coupling between the base 26 and the solar-light power generation unit 80.

The GPS antenna 28 can be configured, in a compact manner, with the base 26 and the conductor 27 that is provided on a surface of the base 26. Because of this, the GPS antenna 28 can be miniaturized. Furthermore, in the plan view in the +Z-axis direction, the GPS antenna 28 and the solar-light power generation unit 80 are arranged in a manner that overlaps with each other. Thus, a light reception surface of the solar-light power generation unit 80 can be increased and a larger amount of generated power can be obtained.

It is noted that the GPS antenna 28 is positioned in a curved manner along the inner circumference of the opening portion 31s of the housing 31 in the twelve o'clock direction (the +Y-axis direction), and that in the plan view in the +Z-axis direction, the GPS antenna 28 is desirably positioned in such a manner as to overlap with the solar-light power generation unit 80 within the second area R2.

The GPS antenna 28 is positioned in the second area R2 that is surrounded with a plurality of operation units (operation buttons) 58, and thus the receiver sensitivity of the GPS antenna 28 can be secured while functions of and an arrangement of the plurality of operation units (operation buttons) 58 are secured.

Furthermore, when viewed from the cross section, that is, from the direction (the X-axis direction) orthogonal to the direction (the +Z-axis direction) normal to the light reception surfaces 80af, 80bf, 80cf, and 80df of the solar-light power generation unit 80, it is preferable that the GPS antenna 28 is positioned between the solar-light power generation unit 80 and the circuit board 20. With this arrangement, the circuit board 20, as well as the base 26, is determined as the dielectric, and thus the wavelength shortening effect can be used and the GPS antenna 28 can be made more compact (small-sized). Furthermore, with the capacity coupling between the GPS antenna 28 and the circuit board 20, the receiver sensitivity of the GPS antenna 28 can be increased.

Furthermore, the GPS antenna 28 is electrically connected to the CPU 21 as the processing unit that is supported on the circuit board 20, via a connection portion 25 that is a contact point which is supported on the circuit board 20 in the conductor 27. With this connection, an electrical connection between the CPU 21 and the GPS antenna 28 that is another member can be made in a compact manner on the circuit board 20, and the level of freedom of an arrangement layout of the GPS antenna 28 and the CPU 21 can be raised.

Furthermore, the conductor 27 does not need to be provided on the entire windshield 55 side (the +Z-axis direction) surface of the base 26, and may be provided on at least one portion of the surface. Furthermore, the conductor 27 may be provided on a flank surface or a rear surface, as well as the windshield 55 side (the +Z-axis direction) surface of the base 26.

It is noted that, although not illustrated, in the plan view in the +Z-axis direction, the GPS antenna 28 may be set to be positioned more outward (to the outer circumference side of the housing 31) than the outer circumference 80os (refer to FIG. 5) of the solar-light power generation unit 80. In other words, in the plan view in the +Z-axis direction, the GPS antenna 28 can be positioned between an inner wall surface 31is of the housing 31, and the first cell 80a and the second cell 80b that constitute the solar-light power generation unit 80. In this manner, in the plan view in the +Z-axis direction, the GPS antenna 28 is positioned between the inner wall surface of the housing and the solar-light power generation unit 80, and thus the receiver sensitivity of the GPS antenna 28 can be secured more effectively.

Constituent elements of the wrist device 200 according to the first embodiment will be further described below, additionally referring to a functional block diagram that is illustrated in FIG. 6.

The circuit board 20 includes a surface 20f (a first surface) and a rear surface 20r (a second surface) that is different from the surface 20f and is a surface that is opposite in direction to the surface 20f. The outer circumference side end portion is attached to a circuit case 75 that is a circuit fixing portion, and is supported inside of the housing 31 via the circuit case 75. The circuit board 20 is positioned on one portion of the first area R1, and in the present embodiment, is positioned in a position that overlaps with the center portion of the first area R1. The angular speed sensor 22 or the acceleration sensor 23 as the sensor that is included in the body motion sensor unit 170, the CPU 21 as the processing unit that includes the control circuit, and the like are mounted on the surface 20f of the circuit board 20 and are electrically connected, and other circuit elements 24 are mounted on the rear surface 20r and are electrically connected. It is noted that, because the outer circumference side of the circuit board 20 is supported on the housing 31, vibration from the housing 31 is easily transferred to the acceleration sensor 23 that is connected to the circuit board 20, and the precision of detection, such as detection of tapping by the acceleration sensor 23 can be much more increased. Furthermore, supported on an end portion of the surface 20f of the circuit board 20 is the connection portion 25 that is the contact point which electrically connects between the GPS antenna 28 and the CPU 21.

Then, the display panel 60 and the solar-light power generation unit 80 are connected to the surface 20f of the circuit board 20 via a connection wiring portion 63 and a connection wiring portion 81, respectively, each of which is configured with a flexible board or the like. Furthermore, the photoelectric sensor unit 40 is electrically connected to the rear surface 20r of the circuit board 20, which is a surface that is opposite in direction to the surface 20f, via a connection wiring portion 46 that is configured with a flexible board or the like. With this arrangement, wire routing for connection can be minimized. Along with this, stray light that refers to incident light for power generation, which becomes leakage light entering from the solar-light power generation unit 80 side and enters a package, can be blocked by the circuit board 20, and thus an influence of external light on the photoelectric sensor unit 40 can be reduced. It is noted that the circuit case 75 can guide the secondary battery 70 or the like.

The angular speed sensor 22 or the acceleration sensor 23 that is included in the body motion sensor unit 170 can detect information relating to motion of the user's body, that is, the body motion information. The angular speed sensor 22 or the acceleration sensor 23 outputs a body motion detection signal that is a signal which changes according to a body motion such as a user's movement or a user's movement in a new direction, and transmits the body motion detection signal that is output, to the CPU 21 as the processing unit that includes the control circuit. It is noted that, for example, detection due to a so-called tapping operation in which the user expresses his/her intention based on an operation by hitting an outer circumference portion of the housing 31, the windshield 55, or the like with his/her fingertip and thus applying a small impact on the housing 31 can be performed as well as detection relating to an operation such as the user's movement. It is noted that a direction sensor (a geomagnetic sensor) and the like can be included in the body motion sensor unit 170.

Here, the angular speed sensor 22 and the acceleration sensor 23 are arranged within the second area R3. Because volumes of elements of the angular speed sensor 22 and the acceleration sensor 23 that are arranged within the second area R3 are small, the angular speed sensor 22 and the acceleration sensor 23 can also be easily arranged in a space-efficient manner in the second areas R2 and R3 in which the solar-light power generation unit 80, the connection wiring portion 63, the GPS antenna 28, and the like are arranged and thus have a comparatively small amount of room for space. It is noted that an example in which the acceleration sensor 23 is positioned in the second area R3 is illustrated in FIG. 5, but that the angular speed sensor 22 and the acceleration sensor 23 may be arranged in either the second area R2 or the second area R3.

Furthermore, it is preferable that at least one portion of the acceleration sensor 23, as illustrated in FIG. 5, is positioned in a position that overlaps with the solar-light power generation unit 80 in the plan view in the +Z-axis direction. In other words, it is preferable that the acceleration sensor 23 is mounted on the very outer circumference side of the circuit board 20, in other words, in a position (the second area R3 that is close to an outer edge portion of the housing 31) that is close to a package inner wall (inner circumference). Alternatively, as illustrated in FIG. 4, it is preferable that a distance between a portion of the circuit case 75, which is brought into contact with the circuit board 20, and the center of the acceleration sensor is shorter than a distance between the center of the acceleration sensor 23 and the center of the circuit board 20. In this manner, by positioning the acceleration sensor 23, an impact on the housing 31, such as the tapping operation is easy to transfer to the acceleration sensor 23 via the circuit board 20 whose outer circumference side is supported on the housing 31, and the precision of the detection by the acceleration sensor 23 can be much more increased.

The CPU 21 as the processing unit is configured with a control circuit or the like that controls a circuit which controls the GPS reception unit 160 which includes the GPS antenna 28, a circuit which drives the photoelectric sensor unit 40 and measures a pulse wave, a circuit which drives the display unit 50 (the display panel 60), a circuit which drives the body motion sensor unit 170, processes the detected signal, and acquires the resulting signal as the body motion information, and a power generation circuit in the solar-light power generation unit 80. Then, the CPU 21 transmits the pulse wave information or the body motion information, which is detected in each corresponding portion, the positional information on the user, or the like to a communication unit 29 whenever necessary.

The GPS antenna 28, along with a signal processing unit 66, is included in the GPS reception unit 160, and receives a plurality of satellite signals. Based on the plurality of satellite signals that are received by the GPS antenna 28, the signal processing unit 66 performs the positioning calculation and acquires a result of the positioning calculation as the positional information on the user.

The communication unit 29 transmits the pulse wave information or the body motion information, or the positional information on the user, which is transmitted from the CPU 21, to the mobile device 300 or the like whenever necessary.

The photoelectric sensor unit 40 as the biological sensor is one that detects a pulse wave or a pulse, and includes a light receiving unit 41 and a plurality of light emitting units 42 (two light emitting units in the present embodiment) that are arranged on both sides of the light receiving unit 41, in other words, more outward (to the outer circumference side of the housing 31) than the light receiving unit 41 in the plan view. In this manner, the light receiving unit 41 is positioned more inward than the light emitting unit 42, and thus external light that enters from the outer circumference side of the housing 31 can be suppressed from entering the light receiving unit 41 and an influence of the external light on the photoelectric sensor unit 40 can be reduced. It is noted that the number of light emitting units 42 is not limited to 2, and may be 1, or 3 or greater. The light receiving unit 41 and two light emitting units 42 are attached to one surface of a sensor board 43, are configured, for example, with photo-curable resin or the like, and are covered with the transparent cover 44 that is configured with a member that transmits light. The transparent cover 44 is inserted into the measurement window portion 45, whose portion includes an area that corresponds to the light receiving unit 41 and the two light emitting units 42 and is provided on the housing 31. It is noted that the transparent cover 44 may protrude from the top of the protrusion portion 32 of the housing 31.

Light that, as described above, is projected from the light emitting unit 42 is emitted to the subject (the measurement target) and reflection light that results from the light being reflected from the subject is received in the light receiving unit 41, and thus the photoelectric sensor unit 40 can detect the pulse wave information. The photoelectric sensor unit 40 outputs a signal that is detected by the pulse wave sensor which includes the light emitting unit 42 and the light receiving unit 41, as a pulse wave detection signal. For example, the photoelectric sensor is used as the photoelectric sensor unit 40. In this case, a technique is considered in which the reflection light or the transmission light, which results from the light emitted from the light emitting unit 42 with respect to the living body (the wrist of the user), is detected by the light receiving unit 41. With this technique, an amount of emitted light absorbed in the living body and an amount of emitted light reflected from the living body vary according to an amount of blood flowing through a blood vessel. Because of this, sensor information that is detected in the photoelectric sensor is a signal that corresponds to the amount of flowing blood or the like. Therefore, information relating to a heartbeat can be acquired by interpreting the signal. However, the pulse wave sensor is not limited to the photoelectric sensor, and any other sensor, such as an electrocardiograph or an ultrasonic sensor, may be used as the pulse wave sensor.

Furthermore, the photoelectric sensor unit 40, as illustrated in FIG. 5, is positioned within the first area R1 that does not overlap with the solar-light power generation unit 80 which is formed in the shape of a ring, in the plan view in the +Z-axis direction. In other words, in the plan view in the +Z-axis direction, the solar-light power generation unit 80 is arranged more outward than an outer edge of the photoelectric sensor unit 40, and is positioned in a position in which the solar-light power generation unit 80 and the photoelectric sensor unit do not overlap with each other. Moreover, in other words, in the plan view in the +Z-axis direction, the solar-light power generation unit 80 is positioned between the bezel 57 and the photoelectric sensor unit 40. Here, it is preferable that the outer edge of the photoelectric sensor unit 40 is an outer edge of an area that includes at least the light receiving unit 41 and outer edges of the two light emitting units 42 with the outer edges being linked with each other, which is indicated by oblique hatching in FIG. 5. It is noted that in the present embodiment, an outer edge of the measurement window portion 45 in which the light receiving unit 41 and the two light emitting units 42 are included can be set to be the outer edge of the photoelectric sensor unit 40. Furthermore, an outer edge of the sensor board 43 may be set to be the outer edge of the photoelectric sensor unit 40. Furthermore, an outer edge of the transparent cover 44 may be set to be the outer edge of the photoelectric sensor unit 40.

In this manner, in the plan view in the +Z-axis direction, the solar-light power generation unit 80 that is positioned in the shape of a ring is positioned more outward than the outer edge of the photoelectric sensor unit 40 in such a manner as to surround the photoelectric sensor unit 40. In other words, a distance over which, in the plan view, the photoelectric sensor unit 40 is positioned in the center portion of the housing 31, or a distance from the photoelectric sensor unit 40 to an inner wall (an inner circumference) of the housing 31 is longer than a distance from the inner circumference 80is of the solar-light power generation unit to an inner wall (an inner circumference) of the housing 31, and thus an influence of external light (leakage light) on the photoelectric sensor unit 40 can be suppressed. Accordingly, because the solar-light power generation unit 80 can be positioned without decreasing the precision of the detection by the photoelectric sensor unit 40, the precision of the detection of the biological information by the photoelectric sensor unit 40 and efficient power generation by the solar-light power generation unit 80 are compatible with each other. Furthermore, in the plan view, the solar-light power generation unit 80 is positioned more outward than the outer edge of the photoelectric sensor unit 40 and the photoelectric sensor unit 40 is positioned in the first area R1. Because of this, in the plan view in the +Z-axis direction, the overlapping of the gap 80s1 between the first cell 80a and the second cell 80b does not take place. Thus, an influence of heat of light that is received by the solar-light power generation unit 80, for example, of solar light, on the photoelectric sensor unit 40 can be reduced. Moreover, the arrangement balance for easily performing the detection by the photoelectric sensor unit 40 while the power generation in the solar-light power generation unit 80 is efficiently performed can be improved, and the property in which the device main body 30 of the wrist device 200 is worn on the user can be improved.

Furthermore, the outer edge of the transparent cover 44 may be the outer edge of the photoelectric sensor unit 40. It is noted that the “non-overlapping” described above refers to a state where S=0 where S is an area of each of the solar-light power generation unit 80 and the photoelectric sensor unit 40 overlapping with each other in the plan view in the +Z-axis direction. Furthermore, the photoelectric sensor unit 40 being surrounded with the solar-light power generation unit 80 can include the photoelectric sensor unit 40 being surrounded with a plurality of solar-light power generation units 80. In some cases, the solar-light power generation unit 80 may be divided or may have a cut. Furthermore, the “overlapping of the biological sensor” refers to a state where the light receiving unit of the biological sensor (the photoelectric sensor) and a constituent element as a target overlap with each other in the plan view in the +Z-axis direction. Alternatively, the “overlapping of the biological sensor” refers to a state where both the light emitting unit and the light receiving unit overlap with each other in the plan view in the +Z-axis direction, or a state where a pulse sensor substrate on which the light transmitting and the light receiving unit are mounted and any other constituent element as a target overlap with each other. Furthermore, the “non-overlapping of the biological sensor” refers to a state where the light receiving unit of the biological sensor (the photoelectric sensor) and any other constituent element do not overlap with each other in in the plan view in the +Z-axis direction. Alternatively, the “non-overlapping of the biological sensor” refers to a state where both the light emitting unit and the light receiving unit do not overlap with a constituent element as a target, in the plan view in the +Z-axis direction, or a state where the pulse sensor substrate on which the light emitting unit and the light receiving unit are mounted and a constituent element as a target do not overlap with each other.

Furthermore, it is preferable that at least one portion of the photoelectric sensor unit 40, as illustrated in FIG. 5, is positioned in such a manner as to overlap with a center G of gravity of the solar-light power generation unit 80 in the plan view in the +Z-axis direction. With this arrangement of the photoelectric sensor unit 40 and the solar-light power generation unit 80, the balance (the center of gravity) of the device main body 30 is satisfactory, and the property of being worn on the user can be improved. It is noted that, in other words, the center G of gravity can be expressed as a center of mass and that in the case of a three-dimensional object, in some cases, the center G of gravity is defined as being within a structure of the three-dimensional object or is defined as being in the space. Furthermore, in some cases, the overlapping of the center G of gravity can be defined as a state where, when viewed from a prescribed direction, the overlapping takes place when the center of gravity is projected on a two-directional plane or a prescribed target object.

The display unit 50 is provided in an area that is positioned more inward toward the center than the inner circumference 80is of the solar-light power generation unit 80 that is positioned in the shape of a ring, via the windshield 55 that is a light transmitting member. The display unit 50 is set to have a configuration in which it is possible that the user visually recognizes the display of a number or an icon which is displayed on a display object, such as the display panel 60, which is provided just under the windshield 55, hands for time displays, or the like, via the windshield 55. More precisely, in the present embodiment, information indicating action information of the users (the biological information or an exercise state), which is derived from results of the detection by various sensors (for example, the acceleration sensor 23), or various pieces of information such as time information are displayed using the display panel 60, and the display is presented from the front surface side (the +Z-axis direction) to the user. It is noted that, as the display object, changing to the display panel 60 that is a liquid crystal display is possible, and an organic electroluminescence (organic EL) display, an electrophoretic display (EPD), alight emitting diode (LED) display, or the like can be used.

The lighting unit 61 functions a backlight of the display panel 60. The lighting unit 61 is connected to the surface 20f (a first surface) of the circuit board 20. The lighting unit 61 is connected in this manner to the circuit board 20, and thus the wire routing for connection can be minimized. Along with this, light that is emitted from the lighting unit 61 can be blocked by the circuit board 20, and an influence of the stray light on the photoelectric sensor unit 40 can be reduced.

Positive and negative terminals of the secondary battery 70 are connected to the circuit board 20 with a connection board (not illustrated), and the secondary battery 70 supplies power to a circuit that controls a power source. Furthermore, the secondary battery 70 is electrically connected to the solar-light power generation unit 80 via the circuit board 20. In this circuit, the power is converted into a prescribed voltage, and so forth, and is supplied to each circuit. Thus, a circuit that drives the photoelectric sensor unit 40 and detects a pulse, and a circuit that drives the display panel 60, a control circuit (the CPU 21) that controls each circuit, and the like are caused to operate. The charging of the secondary battery 70 is performed via one pair charging terminals that is conductively connected to the circuit board 20 by a conducting member (not illustrated) such as a coil spring, or is performed using power that is generated by the solar-light power generation unit 80.

Furthermore, it is preferable that, in the plan view in the +Z-axis direction, the secondary battery 70 is positioned in a position that does not overlap with the solar-light power generation unit 80. In this manner, in the plan view in the +Z-axis direction, the secondary battery 70 is positioned in the position does not overlap with the solar-light power generation unit 80, and thus an influence of heat generation that occurs while charging the secondary battery 70 is difficult to exert on the solar-light power generation unit 80 and a temperature of the solar-light power generation unit 80 can be suppressed from rising. Because of this, the efficiency of power generation in the solar-light power generation unit 80 can be increased.

Using a photovoltaic effect, the solar-light power generation unit 80 converts light energy of external light such as solar light into power and performs power generation. The solar-light power generation unit 80 is divided into four cells for arrangement between the windshield 55 and the display panel 60. A configuration in the so-called ring shape (in the shape of a ring) is employed where the solar-light power generation unit 80 is positioned on an outer circumference portion (the outer edge portion of the display unit 50) that includes an outer edge of the display panel 60, in other words, on the outer circumference side of the housing 31 and where the center portion is a through-hole.

Specifically, the first cell 80a, the second cell 80b, the third cell 80c, and the fourth cell 80d that constitute the solar-light power generation unit 80 are positioned on the opening portion 31s side of the housing 31, have the outer circumference 80os that is an outer edge portion in the shape of a circle, whose length is shorter than an inner circumference length of the opening portion 31s, the inner circumference 80is in the shape of a circle, which is an inner edge portion that is shorter in circumference length than the outer circumference 80os, and two flank sides that connect between one side of the outer circumference 80os and one side of the inner circumference 80is and between the other side of the circumference edge 80os and the other side of the inner circumference 80is, respectively, and are positioned in an outer circumference portion of the display panel 60. That is, each of the cells (the first cell 80a, the second cell 80b, the third cell 80c, and the fourth cell 80d) that have the light reception surfaces 80af, 80bf, 80cf, and 80df, respectively, has an inner circumference that is shorter in circumference length than an outer circumference. It is noted that, in other words, in the plan view in the +Z-axis direction, the shorter radius of a concentric circle that is a shape of the solar-light power generation unit 80 may be the inner circumference and the longer radius may be the outer circumference. The solar-light power generation unit 80 according to the present configuration is configured by arranging four cells that have the light reception surfaces 80af, 80bf, 80cf, and 80df, respectively, along the inner circumference of the opening portion 31s of the housing 31. With the positioning of the solar-light power generation unit 80 in the shape of a ring, a display area of the display unit 50 can be efficiently positioned and so on, and a design property of the wrist device 200 can be increased.

It is noted that in the present configuration, an example is described in which the solar-light power generation unit 80 in the shape of a ring uses four cells (the first cell 80a, the second cell 80b, the third cell 80c, and the fourth cell 80d), but that the solar-light power generation unit 80 may be configured with a plurality of cells and the number of cells does not matter. For example, the solar-light power generation unit 80 may be configured with two cells, one cell that is configured by integrating the first cell 80a and the third cell 80c into one piece without the gap 80s2 between the cells, and the other cell that is configured by integrating the second cell 80b and the fourth cell 80d into one piece without the gap 80s4 between the cells. Furthermore, the solar-light power generation unit 80 may be configured with a film instead of a cell (a panel).

Furthermore, any shapes of the cells (the first cell 80a, the second cell 80b, the third cell 80c, and the fourth cell 80d) that constitute the solar-light power generation unit 80 may be employed as long as the shapes neither damage the visibility of the display unit 50, nor damage the design property thereof. For example, an outer circumference side of each of the cells may be configured with two equal straight lines that result from division in positions along the inner circumference of the opening portion 31s, and outer circumference sides of four cells, as a whole, may be configured with eight equal straight lines that result from division in positions along the inner circumference of the opening portion 31s. Furthermore, an outer circumference side of each of the cells may be configured with three equal straight lines that result from division in positions along the inner circumference of the opening portion 31s, and outer circumference sides of four cells, as a whole, may be configured with twelve equal straight lines that result from division in positions along the inner circumference of the opening portion 31s. Furthermore, it is also possible that the inner circumference of the cell is divided into two equal parts or is divided into three equal parts. Furthermore, it is also possible that the inner circumference and the outer circumference each are divided into two equal parts or are divided into three equal parts. Furthermore, combinations of a cell that is present on the outer circumference or the inner circumference in the shape of a straight line and a cell that is not present can also be used.

Under the control of the CPU 21, the biological information, such as a pulse wave, that is measured by the photoelectric sensor unit 40, the positional information that is measured by the GPS reception unit 160, the body motion information that is measured by the body motion sensor unit 170, and the like are stored in the storage unit 180.

In the wrist device 200, as the mobile electronic device, according to the first embodiment, which is described above, with power that is generated by the solar-light power generation unit 80 that has at least the first cell 80a and the second cell 80b which are arranged with the gap 80s1 in between, position measuring, heartbeat measuring, information communication, or the like can be performed. Because of this, under various use environments, power to use can also be provided by self-power-generation, or the frequency of charging can also be decreased.

In addition, in the plan view, the gap 80s1 between the first cell 80a and the second cell 80b, the mark (the first mark 90) that is provided on the windshield 55 as the light transmitting member, and the flexible wiring board 65 as the wiring portion are arranged in a manner that overlaps with each other, in the second area R2 that is different from the first area R1 which is an area that is surrounded with a plurality of operation units (operation button) 58, for example, an area that is defined by connecting the protrusion portion that protrudes toward the sides of the plurality of operation units (operation buttons) 58, which face the external flank surface of the device main body 30, with straight lines. Because of this, the width (the area) or the space (the volume) of the first area R1 in which the circuit board 20 is positioned can be sufficiently secured, and the miniaturization and the thinning of the housing 31 (the wrist device 200) can be realized.

Furthermore, in the plan view in the +Z-axis direction, the acceleration sensor 23 is positioned in a position that does not overlap with the gap 80s1 between the first cell 80a and the second cell 80b. Because of this, it is difficult for solar light that enters from the gap 80s1 to reach the acceleration sensor 23, and a characteristic change that results from a change in the temperature of the acceleration sensor 23 due to the solar light can be reduced.

### 4. 2 Wrist Device According to a Second Embodiment

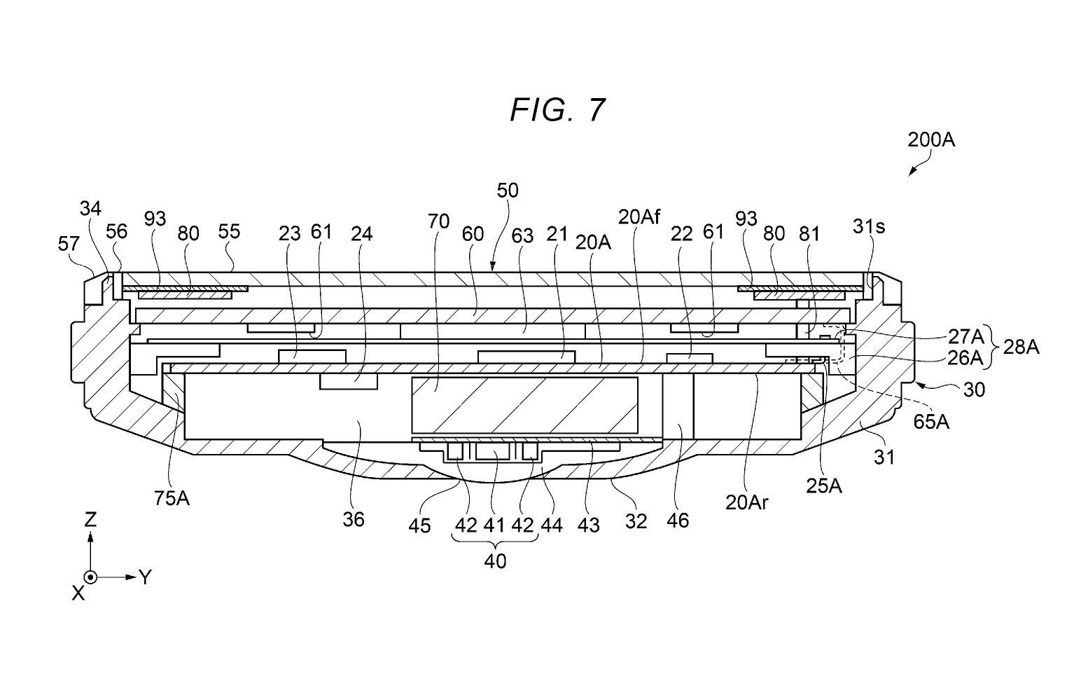

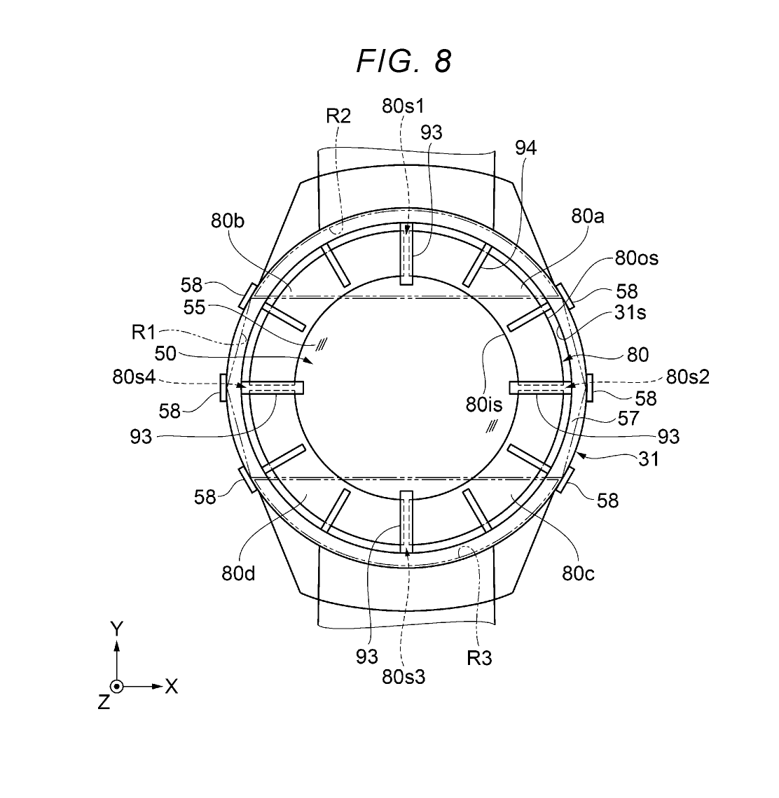

Next, a configuration of the wrist device (the measurement device), as the mobile electronic device, according to a second embodiment will be described with reference to FIGS. 7 and 8. FIG. 7 is a cross-sectional diagram illustrating the configuration of the wrist device according to the second embodiment. FIG. 8 is a plan-view diagram illustrating the configuration of the wrist device according to the second embodiment. It is noted that in the following description relating to the second embodiment, a configuration that is different from that according to the first embodiment, which is described above, is emphatically described, and that the same configuration is given the same numeral reference in each figure and a description thereof is omitted.

Furthermore, in the following description, in the same manner as in the first embodiment, in the plan view in the +Z-axis direction, an area that is the center portion of the housing 31, which is defined by connecting protrusion portions that protrudes toward sides of a plurality of operation units (operation buttons) 58 that are connected to a circuit board 20A (refer to FIG. 7), which face an external flank surface of the device main body 30, with straight lines, is defined as a first area R1. Furthermore, areas that are different from the first area R1, which overlap with the housing 31, are defined as second areas R2 and R3. In this configuration, an area that is positioned in the +Y-axis direction (toward the twelve o'clock side) with respect to the first area R1 is expressed as a “second area R2,” and an area that is positioned in the −Y-axis direction (toward the six o'clock side) with respect to the first area R1 is expressed as a “second area R3.”

The wrist device 200A according to the second embodiment, which is illustrated in FIGS. 7 and 8 is different from the wrist device 200 according to the first embodiment, which is described above, in terms of a configuration and positioning of a GPS antenna 28A and configurations and positioning of marks (a time display mark 93 and a time auxiliary display mark 94) that is provided on the windshield 55, and is accordingly different from the first embodiment in terms of a support configuration of a circuit board 20A. Configurations other than these are the same as those of the constituent components of the wrist device 200 according to the first embodiment. In the following description, the GPS antenna 28A that has a different configuration, the support configuration of the circuit board 20A, and the marks (the time display mark 93 and the time auxiliary display mark 94) on the windshield 55 are emphatically described.

In the same manner as in the first embodiment, the device main body 30 of the wrist device 200A according to the second embodiment has the housing 31 including the opening portion 31s which is open to the front side, and the measurement window portion 45 of the photoelectric sensor unit 40 is provided on the rear side. The opening portion 31s of the housing 31 is closed by the windshield 55 as the light transmitting member that protects the internal structure. Accordingly, the inside of the housing 31 is the internal space 36 that is a closed space which is surrounded with the housing 31 and the windshield 55. Then, as illustrated in FIG. 7, constituent elements that constitute the wrist device 200A are accommodated in the internal space 36 within the housing 31, and this is the same as in the first embodiment. Thus, detailed descriptions thereof the constituent elements are omitted.

The GPS antenna 28A as the antenna unit includes a base 26A, as a non-conductive member, which is configured, for example, with a resin member, and a conductor 27A that is configured, for example, with metal such as copper, copper alloy, aluminum, or aluminum alloy, which is provided on the windshield 55 side (the +Z-axis direction) surface of the base 26A. The base 26A has a shape, such as the shape of a ring along the inner circumference of the opening portion 31s of the housing 31 or the shape of a ring that results from combining arc-shaped members along the inner circumference of the opening portion 31s, and is fixed to the inner wall of the housing 31. It is noted that, in the plan view in the direction (the +Z-axis direction) normal to the light reception surfaces 80af, 80bf, 80cf, and 80df of the solar-light power generation unit 80, the base 26A is positioned within the first area R1 in such a manner as to overlap with the solar-light power generation unit 80. The conductor 27A is configured with a sheet metal that is positioned between the display panel 60 and the circuit board 20A and has the shape of roughly a disk facing the circuit board 20A, and an outer circumference portion of the conductor 27A is connected to the windshield 55 (the +Z-axis direction) side surface of the base 26A. The non-conducting base 26A is determined as a dielectric, and thus wavelength shortening effect due to the base 26A (the dielectric) is used. As a result, the GPS antenna 28A is configured as an antenna. The antenna can be thinly configured with the base 26A and the conductor 27A that is positioned to include the base 26A.

It is noted that, in the plan view in the +Z-axis direction, the base 26A which constitutes the GPS antenna 28A is desirably positioned around a contour of the display unit 50, in other words, more outward (to the outer circumference side of the housing 31) than the inner circumference 80is of the solar-light power generation unit 80 that will be described below, and in the first area R1. In this manner, if the base 26A is positioned to the outer circumference side of the housing 31 and in the first area R1, the level of freedom of an arrangement layout of the display unit 50, the solar-light power generation unit 80 or the like can be raised, and the display unit 50, the solar-light power generation unit 80, or the like can be more efficiently positioned.

Furthermore, the GPS antenna 28A is electrically connected to the CPU 21 that is supported on the circuit board 20A, via the connection portion 25A that is mounted on the circuit board 20A. With this connection, an electrical connection between the CPU 21 and the GPS antenna 28A that is another member can be made in a compact manner on the circuit board 20A. Then, in the plan view in the +Z-axis direction, the connection portion 25A is desirably positioned in a manner that overlaps with the solar-light power generation unit 80, and thus the same effect as in the first embodiment can be achieved.

Furthermore, the conductor 27A may be provided on the surface 20Af side of the circuit board 20A that is positioned on the windshield 55 side (the +Z-axis direction) of the base 26A, but also on the rear surface 20A side that is opposite in direction to the surface 20Af side. Furthermore, the conductor 27A may be provided on both sides, the surface 20Af side and the rear surface 20Ar side.

The circuit board 20A includes a surface 20Af (a first surface) and a rear surface 20Ar (a second surface) that is a surface that is opposite indirection to the surface 20Af. The outer circumference side end portion is attached to a circuit case 75A that is a circuit fixing portion, and is supported inside of the housing 31 via the circuit case 75A. The angular speed sensor 22 or the acceleration sensor 23 as the sensor that is included in the body motion sensor unit 170, the CPU 21 as the processing unit that includes the control circuit, and the like are mounted on the surface 20Af of the circuit board 20A and are electrically connected, and other circuit element 24 is mounted on the rear surface 20Ar and is electrically connected. Furthermore, supported (connected) on an end portion of the surface 20Af of the circuit board 20A is the connection portion 25A that is the contact point which electrically connects between the GPS antenna 28A and the CPU 21. Furthermore, connected to the circuit board 20A is a flexible wiring board 65A as a wiring unit that electrically connects between the circuit board 20A and the display panel 60.

The flexible wiring board 65A electrically connects between the circuit board 20A and the display panel 60. In the plan view in the +Z-axis direction, the flexible wiring board 65A is provided in the second area R2 in the +Y-axis direction. It is noted that in the plan view in the +Z-axis direction, the flexible wiring board 65A may be positioned in such a manner as to overlap with the gap 80s1 between the first cell 80a and the second cell 80b. Furthermore, in the plan view in the +Z-axis direction, the flexible wiring board 65A may be positioned in the second area R2 that does not overlap with the gap 80s1 between the first cell 80a and the second cell 80b. In this manner, the flexible wiring board 65A is positioned in the second area R2, and thus, the aesthetical property and the easiness of design can be secured while a function of the operation unit (the operation button) 58 is maintained.

The solar-light power generation unit (solar cells) 80 is divided into four cells (the first cell 80a, the second cell 80b, the third cell 80c, and the fourth cell 80d) between the windshield 55 and the display panel 60, and is positioned in such a manner that gaps 80s1, 80s2, 80s3, and 80s4 are present between pairs of the cells, respectively. It is noted that a specific arrangement of and configurations of the four cells (the first cell 80a, the second cell 80b, the third cell 80c, the fourth cell 80d) are the same as those in the first embodiment, and thus descriptions thereof are omitted.

Furthermore, marks (the time display mark 93 and the time auxiliary display mark 94) that are positioned on the outer circumference side of the windshield 55 and are expressed in an approximately straight line that extends radially from the center of the housing 31 toward outward are provided on the rear surface side of the windshield 55. Time display marks 93 indicate positions, respectively, of 12 o'clock, 3 o'clock, 6 o'clock and 9 o'clock. The time display marks 93 are configured to include at least planar shapes, respectively, in the +Z-axis direction, of the gaps 80s1, 80s2, 80s3, and 80s4 that are provided between pairs of cells (the first cell 80a, the second cell 80b, the third cell 80c, and the fourth cell 80d), respectively, that constitute the solar-light power generation unit 80. In this manner, the marks (the time display marks 93) in shapes (the four gaps, the gaps 80s1, 80s2, 80s3, and 80s4 in the present embodiment) that include at least the planar shape of the gap 80s1 between the firs cell 80a and the second cell 80b are provided, and thus, in the plan view in the +Z-axis direction, the four gaps, the gaps 80s1, 80s2, 80s3, and 80s4 are difficult to visually recognize.

It is noted that a mark other than the time display mark 93 and the time auxiliary display mark 94 may be provided on the rear surface side of the windshield 55.

In the wrist device 200A, as the mobile electronic device, according to the second embodiment, which is described above, with power that is generated by the solar-light power generation unit 80 that has at least the first cell 80a and the second cell 80b which are arranged with the gap 80s1 in between, the position measuring, the heartbeat measuring, the information communication, or the like can be performed. Because of this, under various use environments, power to use can also be provided by self-power-generation, or the frequency of charging can also be decreased.

In addition, in the plan view, the marks (the time display marks 93) in shapes (the four gaps, the gaps 80s1, 80s2, 80s3, and 80s4 in the present embodiment) that include at least the planar shape of the gap 80s1 between the firs cell 80a and the second cell 80b are provided, and thus, in the plan view in the +Z-axis direction, the four gaps, the gaps 80s1, 80s2, 80s3, and 80s4 are difficult to visually recognize.

Furthermore, the base 26A as the non-conductive member is determined as a dielectric, and thus the wavelength shortening effect due to the base 26A is used. As a result, with the base 26A and the sheet-metal conductor 27A that is provided on the surface of the base 26A, the GPS antenna 28A that is thinly configured can be realized. Then, in the plan view in the +Z-axis direction, at least one portion of the base 26A of the GPS antenna 28A is positioned in a manner that overlaps with the solar-light power generation unit 80. Because of this, in the wrist device 200A as a small-sized mobile electronic device, it is also possible that constituent elements such as the solar-light power generation unit 80 and the GPS antenna 28A are accommodated within the housing 31.

It is noted that in the embodiment, as an example of the positioning system that uses the positional information satellite, the GPS that uses the GPS satellite 8 as the positional information satellite, which is included in the global navigation satellite system (GNSS), is described above, and that this is only an example. The global navigation satellite system may be any other system, such as a GALILEO (EU), GLONASS (RUSSIA), BEIBOU (CHINA), or may be one that includes a stationary satellite such as SBAS, or a positional information satellite which transmits the satellite signal, such as a quasi-zenith satellite. That is, the wrist device 200 may be configured to acquire any one of date information, time information, positional information, and speed information, which is known by processing a radio wave (a wireless signal) from a positional information satellite other than the GPS satellite 8. It is noted that the global navigation satellite system can be set to be a Regional Navigation Satellite System (RNSS).

What is claimed is:

1. A mobile electronic device comprising:
a housing that is worn on a body of a user;
a solar-light power generation unit that is accommodated in the housing and has a first cell and a second cell that are arranged with a gap in between;
a display that is accommodated in the housing;
a plurality of buttons on which an operation by the user is applied;
a circuit board that is accommodated in the housing, is electrically connected to the solar-light power generation unit, and overlaps, in a plan view in a direction normal to a light reception surface of the first cell, with at least one portion of a first area that is configured by connecting the plurality of buttons with straight lines;
a wiring unit that is positioned in a second area which is different from the first area in the plan view and includes the gap;
a biological sensor that is accommodated in the housing, is positioned in a position that overlaps with the first area, and detects a biological signal of the user; and
a wiring unit that electrically connects between the solar-light power generation unit which is positioned in a second area which is different from the first area in the plan view and includes the gap, and the circuit board.

2. The mobile electronic device according to claim 1, further comprising:
a light transmitting member that is positioned on the light reception surface side of the solar-light power generation unit and has a mark which overlaps with the gap in the plan view,
wherein the gap, the mark and the wiring unit overlap with one another in the plan view.

3. The mobile electronic device according to claim 2,
wherein the mark includes at least any one of a letter, a symbol, and a line.

4. The mobile electronic device according to claim 2,
wherein the mark has a shape that includes at least a planar shape of the gap in the plan view.

5. The mobile electronic device according to claim 1,
wherein the solar-light power generation unit overlaps with an outer edge of the display unit in the plan view.

6. The mobile electronic device according to claim 1, further comprising:
an antenna unit that is accommodated in the housing, is electrically connected with the circuit board, and receives a wireless signal,
wherein the antenna unit and the solar-light power generation unit overlap with each other in the plan view.

7. The mobile electronic device according to claim 1, further comprising:
an antenna unit that is accommodated in the housing, is electrically connected to the circuit board, and receives a wireless signal,
wherein the antenna unit is positioned between an internal wall surface of the housing and the solar-light power generation unit in the plan view.

8. The mobile electronic device according to claim 1, further comprising:
an antenna unit that is accommodated in the housing, is electrically connected to the circuit board, and receives a wireless signal,
wherein the antenna unit is positioned in the second area.

9. The mobile electronic device according to claim 1, further comprising:
an acceleration sensor that detects a body motion of the user,
wherein the acceleration sensor is positioned in the second area.

10. A mobile electronic device comprising:
a housing that is worn on a body of a user;
a solar-light power generation unit that is accommodated in the housing and has a first cell and a second cell that are arranged with a gap in between;
a biological sensor that is accommodated in the housing and detects a biological signal of the user;
a display unit that is accommodated in the housing, and on which biological information on the user, which is derived based on a signal from the biological sensor, is displayed; and
a circuit board that is accommodated in the housing, and is electrically connected to the solar-light power generation unit, the biological sensor, and the display unit,
wherein the gap in the solar-light power generation unit and the biological sensor do not overlap with each other in a plan view in a direction normal to a light reception surface of the first cell.

11. The mobile electronic device according to claim 10,
wherein in a cross sectional view in a direction orthogonal to a direction normal to the light reception surface of the first cell and a light reception surface of the second cell, the display unit is positioned between the solar-light power generation unit and the circuit board.

12. The mobile electronic device according to claim 10, further comprising:
a biological sensor that detects the biological signal of the user,
wherein in the cross sectional view, the circuit board is positioned between the biological sensor and the solar-light power generation unit.

13. The mobile electronic device according to claim 12, further comprising:
a secondary battery that supplies power to at least the circuit board and the biological sensor,
wherein in the cross sectional view, the secondary battery is positioned between the biological sensor and the circuit board.

14. The mobile electronic device according to claim 10, further comprising:
a plurality of buttons that are connected to the circuit board and on which an operation of the user is applied; and
a wiring unit that electrically connects between the circuit board and the display unit and is positioned to include the gap in the plan view,
wherein in the plan view, the circuit board has a first area that is configured by connecting the plurality of buttons with straight lines,
wherein the biological sensor is positioned in the first area, and
wherein in the plan view, the wiring unit is positioned in a second area that is different from the first area.

15. The mobile electronic device according to claim 14,
wherein an acceleration sensor that detects a body motion of the user is positioned in the second area.
